(19) United States
(12) Patent Application Publication
(10) Pub. No.: US 2019/0338007 A1
(43) Pub. Date: Nov. 7, 2019

(54) METHODS AND MEANS FOR EXPRESSION OF AUTHENTIC AND BIOACTIVE BASIC FIBROBLAST GROWTH FACTOR IN BACILLUS SUBTILIS

(71) Applicant: Chui Chi LAM, Kowloon (HK)

(72) Inventors: Wan Keung Raymond Wong, Kowloon (HK); Xiu Hua Hu, Kowloon (HK); Ka Lun Ng, Kowloon (HK); Chui Chi Lam, Kowloon (HK)

(73) Assignee: Chui Chi LAM, Kowloon (HK)

(21) Appl. No.: 16/401,858

(22) Filed: May 2, 2019

(30) Foreign Application Priority Data
May 7, 2018 (HK) HK18105835.8

(51) Int. Cl. C07K 14/50; C12R 1/125

(52) U.S. Cl. CPC C07K 14/50; C12R 1/125

(57) ABSTRACT

The present invention is concerned with a method of production of authentic and bioactive human basic fibroblast growth factor (bFGF) of 146 amino acids and without any modification at either C- or N-terminal of the bFGF. The method includes the steps as of providing a Bacillus subtilis host, introducing a DNA construct into the Bacillus subtilis host to produce a transformed Bacillus subtilis host, the DNA construct including an insert consisting of, from 5′ to 3′, a cellulose binding domain (CellBD), an intein sequence and a DNA coding for the bFGF polypeptide, and subjecting the transformed Bacillus subtilis host to a shake flask cultivation process or a fed-batch fermentation process.

### RELATED APPLICATION

The present application claims priority from earlier filed Hong Kong application no. 18105835.8 filed on May 7, 2018, contents of which are incorporated herein in their entirety.

### SEQUENCE LISTING

The instant application contains a Sequence Listing which has been submitted electronically in ASCII format and is hereby incorporated by reference in its entirety. Said ASCII copy, created on Mar. 15, 2019, is named G02-008A 190315 Sequence listing.txt and is 5 KB in size.

### FIELD OF THE INVENTION

The present invention is concerned with expression of authentic and bioactive basic fibroblast growth factor (bFGF) in Bacillus subtilis, the bFGF having 146 amino acid residues.

### BACKGROUND OF THE INVENTION

Basic fibroblast growth factor is a functionally versatile but currently very expensive polypeptide. While there had been proposals in the past in the production of the polypeptide, many of such proposals were not concerned with producing authentic and bioactive fibroblast growth factor which has the native characteristics. In other words, many such proposals were only able to generate analogs or isoforms which do not behave similarly as the native polypeptide or do not have comparable efficacy. In some cases, there were also issues with safety arising from production of the polypeptide or analogs. For example, some of the proposals made use of biological systems which would generate undesired side products, e.g. toxics, rending the isolation of the human basic fibroblast growth factor not suitable for human application.

On the other hand, cost-effective production of recombinant proteins is a prerequisite for the widespread availability of the products on the market. Basic fibroblast growth factor, notwithstanding a versatile protein shown to play important functions in various physiological processes including angiogenesis, wound healing and chondrogenesis, has not been commonly applied as expected. For example, authentic human basic fibroblast growth factor (bFGF) is a 16.5 kDa protein comprising 146 amino acid residues (SEQ ID NO. 1-NH₂PALPEDGGSG^{|10}AFPPGHFKDP^{|20}KRLYCKNGGF^{|30}FLRIHPDGRV^{|40}DGVREKSDPH^{|50}IKLQ LQAEER^{|60}GVVSIKGVCA^{|70}NRYLAMKED^{|80}GRLLASKCVT^{|90}DECFFFERLE^{|100}SNNYNTYRSR^{|110}KYTSWYVALK^{|120}RTGQYKLGSK^{|130}TGPGQKAILFL^{|140}PMSAKS-COOH). However, essentially only structural analogs of bFGF of various molecular sizes are available for commercial applications. The reason is probably due to the use of conventional cloning methods, which are unable to establish a cost-effective processing protocol, to result in bFGF. Thus, bFGF has not been commonly available for skin care or therapeutic applications. Incredibly, however, despite being unauthentic, bFGF analogs are sold at extremely high prices, ranging from approximately US$1,300 to US$2,000 per mg at the time of filing of this application. Thus, only cost-effective availability of bFGF on the market may help lower its unreasonably high prices.

The present invention seeks to address the aforementioned issues, or at least to provide an alternative to the public.

### SUMMARY OF THE INVENTION

According to a first aspect of the present invention, there is provided a method of production of authentic and bioactive human basic fibroblast growth factor (bFGF) of 146 amino acids intracellularly and without any modification at either C- or N-terminal of the bFGF, comprising the steps of providing a Bacillus subtilis host, introducing a DNA construct into the Bacillus subtilis host to produce a transformed Bacillus subtilis host, the DNA construct including an insert consisting of, from 5′ to 3′, a cellulose binding domain (CellBD), an intein sequence and a DNA coding for the bFGF polypeptide, and subjecting the transformed Bacillus subtilis host to a shake flask cultivation process or a fed-batch fermentation process, whereby to enable the transformed Bacillus subtilis host to produce the bFGF in a soluble form, cleaved, independent from proteins encoded by DNA regions preceding and subsequent to the bFGF DNA coding in the insert, and intracellularly.

Preferably, the intein sequence may be Ssp DnaB.

Suitable, the method, when subjected to the shake flask cultivation process may comprise a step of using a shake flask with a 100 ml of culturing medium, or when subjected to the fed-batch fermentation process, may comprise a step of using a fermentor with a 2-L of culturing medium. The production of the bFGF via the fed-batch fermentation process may be more than twice as much as via the shake flask cultivation process.

According to a second aspect of the present invention, there is provided a method of production of authentic and bioactive human basic fibroblast growth factor (bFGF) of 146 amino acids and without any modification at either C- or N-terminal of the bFGF, comprising the steps of providing a Bacillus subtilis host, introducing a DNA construct into the Bacillus subtilis host to produce a transformed Bacillus subtilis host, the DNA construct including an insert consisting of, from 5′ to 3′, an intein sequence and a DNA coding for the bFGF polypeptide, and subjecting the transformed Bacillus subtilis host to a shake flask cultivation process or a fed-batch fermentation process, whereby to enable the transformed Bacillus subtilis host to produce the bFGF in a soluble form cleaved and independent from proteins encoded by DNA regions preceding and subsequent to the bFGF DNA coding in the insert. Preferably, the insert may consist of, from 5′ to 3′, a cellulose binding domain (CellBD), the intein sequence and the DNA coding for the bFGF polypeptide.

According to a third aspect of the present invention, there is provided a method of production of authentic and bioactive polypeptide intracellularly and without any modification at either C- or N-terminal of the polypeptide, comprising the steps of providing a Bacillus subtilis host, introducing a DNA construct into the Bacillus subtilis host to produce a transformed Bacillus subtilis host, the DNA construct including an insert consisting of, from 5′ to 3′, a cellulose binding domain (CellBD), an intein sequence and a DNA coding for the polypeptide, and subjecting the transformed Bacillus subtilis host to a shake flask cultivation process or a fed-batch fermentation process, whereby to enable the transformed Bacillus subtilis host to produce the polypeptide in a soluble form, cleaved, independent from proteins encoded by DNA regions preceding and subsequent to the DNA coding in the insert, and intracellularly.

According to a fourth aspect of the present invention, there is provided a biological system engineered from a Bacillus subtilis host, comprising a DNA construct including an insert consisting of, from 5′ to 3′, a cellulose binding domain (CellBD), an intein sequence and a DNA coding for the bFGF polypeptide. Preferably, the intein sequence may be Ssp DnaB.

According to a fifth aspect of the present invention, there is provided a biological system engineered from a Bacillus subtilis host, comprising a DNA construct including an insert consisting of, from 5′ to 3′, an intein sequence and a DNA coding for the bFGF polypeptide. The insert may consist of, from 5′ to 3′, a cellulose binding domain (CellBD), the intein sequence and the DNA coding for the bFGF polypeptide.

According to a sixth aspect of the present invention, there is provided a DNA construct comprising an insert consisting of, from 5′ to 3′, a cellulose binding domain (CellBD), an intein sequence and a DNA coding for the bFGF polypeptide. The intein sequence may be Ssp DnaB.

According to a seventh aspect of the present invention, there is provided a DNA construct comprising an insert consisting of, from 5′ to 3′, an intein sequence and a DNA coding for the bFGF polypeptide. The insert may consist of, from 5′ to 3′, a cellulose binding domain (CellBD), the intein sequence and the DNA coding for the bFGF polypeptide.

### BRIEF DESCRIPTION OF THE DRAWINGS

Some embodiments of the present invention will now be explained, with reference to the accompanied drawings, in which:

FIGS. 1A & 1B are a schematic representation of DNA constructs with an insert expressing the intein, DnaB, fused with different protein molecules (The top diagram, i.e. FIG. 1A, shows vector pM2Veg (5.7 kb) with different DNA coding sequences inserted as shown below to form plasmids: (A) pM2-DnaB-bFGF (6.5 kb) and (B) pM2-CellBD-DnaB-bFGF (6.8 kb), which were expected to result in the expression of precursor/intermediate products: DnaB-bFGF and CellBD-DnaB-bFGF, respectively. Symbols for genetic elements shown in pM2Veg and its derivatives are: ori=replication origin of B. subtilis; neo=structural gene conferring resistance to neomycin; bFGF=bFGF gene; VegC=vegC promoter; lacO=lac operator. Arrows indicate directions of gene expression);

FIGS. 2A & 2B are gel images of a Western blot analysis of recombinant bFGF expressed in lysate samples of B. subtilis cultures (Culture samples of (A) B. subtilis [pM2-DnaB-bFGF] and (B) B. subtilis [pM2-CellBD-DnaB-bFGF] grown under IPTG induction were collected from different time intervals and analyzed. Lane +ve: bFGF standard (0.5 μg); lanes 12 h, 16 h and 20 h: samples collected from cultures induced for 12 h, 16 h, 20 h, respectively; each lane was loaded with 10 μl of cell culture);

FIGS. 3A & 3B include a gel image and a graph of a time course study of bFGF expression in Bacillus subtilis [pM2-CellBD-DnaB-bFGF] cells grown in shake flask (Samples were collected from the culture grown under IPTG induction at different time intervals. (A) Western blotting of recombinant bFGF present in lysate samples. All the wells were loaded with a sample size equivalent to 10 μl of the cell culture. (B) Quantitative analysis of bFGF and cell viabilities. Levels of bFGF detected in the cell lysates ( [symbol] ) are presented. Viabilities of plasmid free and plasmid containing cells were determined on plain agar plates ( [symbol] ) and plates supplemented with kanamycin ( [symbol] ) respectively. CFU refers to colony-forming units. Growth experiment of the transformant was repeated three times and standard error bars are shown);

FIGS. 4A & 4B include a gel image and graph of a time course study of bFGF expression in Bacillus subtilis [pM2-CellBD-DnaB-bFGF] cells cultivated in a 2 L fermentor ((A) Western blotting of recombinant bFGF present in lysate samples. All the wells were loaded with a sample size equivalent to 5 μl of the cell culture. (B) Quantitative analysis of bFGF and cell viabilities. Samples were collected from the culture grown under IPTG induction at different time points. Levels of bFGF detected in the cell lysates ( [symbol] ) are presented. Viabilities of plasmid free and plasmid containing cells were determined on plain agar plates ( [symbol] ) and plates supplemented with kanamycin ( [symbol] ), respectively. CFU refers to colony-forming units. Growth experiment of the transformant was repeated three times and standard error bars are shown); and

FIGS. 5A, 5B & 5C include gel images and a graph showing mitogenicity of recombinant bFGF (Details of purification of bFGF from cell lysates and the assay for mitogenic effects of bFGF on the proliferation of BALB/C 3T3 fibroblast cells were described in Materials and methods. Western blot analysis with antibodies raised against: (A) FRS2α; (B) phosphotyrosine. The two blots contain the same arrangement of bFGF samples. Lane 311ROmpAd: bFGF purified from E. coli [pWK311ROmpAd] culture (Kwong et al. 2016b); Lane CellBD-DnaB-FGF: bFGF purified from B. subtilis [pM2-CellBD-DnaB-bFGF] culture; Lane −ve: buffer without bFGF. (C) Comparison of mitogenic effects exhibited by different concentrations of purified bFGF samples derived from E. coli [pWK-311ROmpAd] ( [symbol] ) and B. subtilis [pM2-CellBD-DnaB-bFGF] ( [symbol] ) cultures. The assay was repeated four times and standard error bars are shown).

### DETAILED DESCRIPTION OF PREFERRED EMBODIMENTS OF THE INVENTION

Expression of desirable proteins by making use of E. coli as a host has been proposed in different contexts. However, this Gram-negative bacterium suffers from the drawbacks of being an endotoxin producer and susceptible to high cell lethality due to plasmid curing. As a result, these undesirable effects could make growth studies more complicated and difficult.

During the course leading to the present invention, the inventors have identified that, under certain conditions, Bacillus subtilis is an alternative host for recombinant protein expression of bFGF, and is an alternatively host for recombinant protein expression intracellularly. This is unexpected for a number of reasons. First, compared to other hosts such as E. coli, Bacillus subtilis is not as well characterized. Nevertheless, B. subtilis, being a Gram-positive bacterium, is free of endotoxins, and is thus recognized as a GRAS (generally recognized as safe) organism. In addition, recombinant B. subtilis strains have been shown to yield stable growth, thus making optimization of product expression comparatively less complicated. Nevertheless, B. subtilis would be able to yield high levels of homologous proteins, e.g. α-amylase, in the past there was little or no demonstration to use B. subtilis to achieve high-level expression of heterologous proteins in this bacterium.

Over 600 putative intein genes have been proposed and many of them have been evaluated for use in mediating gene expression, essentially using E. coli as the host. Previous findings showed that intein-mediated intracellular expression had been successful in resulting efficient production of proteins possessing authentic structures in E. coli but there was no suggestion that inteins could be used in other circumstances. Secretory expression was limited by the efficacy of the secretion signal employed as well as well as the channels available for transportation. During the course leading to the present invention, efforts were made to attempt possibilities which not only were not conducted before, but were also thought to be not feasible. The present invention makes of B. subtilis as a biological host for expression of a heterologous proteins, namely bFGF, and that the bacterium does not produce endotoxins, rendering the produced bFGF safe for use.

The present invention provides the development of a B. subtilis intracellular expression system, which employs the intein, Ssp DnaB, along with an endoglucanase cellulose binding domain to facilitate successful production of bFGF as a soluble and precisely processed mature protein in the cytoplasm of B. subtilis. In addition, scale-up production of bFGF in fermentors showed that the recombinant culture maintained high levels of cell viability and plasmid stability, thus enabling a substantial improvement (˜170%) of yield of bFGF. By improvement, it refers to comparison between the yield obtained in shake flasks (31 mg/L) and that in a 2 L fermentor (84 mg/L). Please see below description. The findings support that the described B. subtilis intein-mediate expression approach provides a practical solution for the production of toxin-free bFGF, and foreseeably, other medically valuable proteins.

Inteins, also known as “protein introns”, have been found to be present in many microbial species and widely employed for the expression and purification of recombinant proteins only in Escherichia coli. The present invention has demonstrated that, despite the likelihood of absence of inteins in B. subtilis, this bacterium is able to facilitate auto-catalytic cleavages of fusions formed between inteins and recombinant proteins. Employing a construct expressing the intein, Ssp DnaB, (DnaB), which was fused at its N-terminus with the cellulose binding domain (CellBD) of an endoglucanase encoded by the cenA gene of Cellulomonas fimi, the construct was demonstrated to be capable of mediating intracellular expression of basic fibroblast growth factor (bFGF), followed by auto-processing of the CellBD-DnaB-bFGF fusion to result in bFGF possessing the 146 amino acid authentic structure. The mentioned fusion was shown to result in a high yield of 84 mg l⁻¹ of biologically active bFGF. Future work in improving the growth of B. subtilis may enable the use of this bacterium, working in cooperation with inteins, to result in a new platform for efficient expression of valuable proteins.

The present invention is further illustrated by way of the below experiments, results and discussions

Materials and Methods

Bacterial Strains and Chemicals

E. coli strains ER2925 (NEB; Ipswich, Mass., USA) and JM101 (Sivakesava et al. 1999) were used as intermediate hosts for recombinant DNA manipulations. B. subtilis strain 1A751 was described previously (Kwong et al. 2013a). The Phusion PCR Kit, restriction and modifying enzymes were purchased from NEB (Ipswich, Mass., USA). All Oligos were purchased from Invitrogen (Carlsbad, Calif., USA.) Chemicals used in this study were purchased from Sigma-Aldrich Corporation (St. Louis, Mo., USA) unless otherwise specified. Antibodies against bFGF were raised in rabbits.

Engineering of Constructs Expressing Fusions Comprising Intein Ssp DnaB and bFGF

The engineering of construct pM2-DnaB-bFGF was achieved using many steps and rounds of overlap extension PCR summarized as follows. Firstly, with oligos P1 and P2 (Table 1) as primers and a derivative of plasmid pM2VegCenA (Lam et al. 1998), pFC, which was extended by PCR to regain the 5′-terminal 1-45 codons (deleted in pM2VegCenA) of the full-length cenA gene (Wong et al. 1986), as the template, a fragment comprising the vegC promoter, lac operator and RBS of B. subtilis (Product 1-1) was attained. Secondly, using oligos P3 and P4 (Table 1) as primers and plasmid pTWIN1 (NEB; Ipswich, Mass., USA) as the template, a sequence with the intein gene for Ssp DnaB and nucleotides overlapping those at (i) the 3′end of Product 1-1, and (ii) the 5′ end of the coding sequence for bFGF (Product 1-2), was obtained. Thirdly, with oligos P5 and P6 (Table 1) as primers and pWK3R (Kwong et al. 2013b) as the template, a fragment containing the coding sequence for bFGF fused with a partial sequence of Ssp DnaB (Product 1-3) was generated. All Products were purified and subjected to second rounds of overlap extension PCR. To obtain a precise fusion (Product 2-1) between the sequences coding for Ssp DnaB and bFGF, oligos P3 and P6 (Table 1) were used as primers, while Product 1-2 and Product 1-3 were employed as templates. Similarly, using oligos P1 and P6 (Table 1) as primers, along with Products 1-1 and 2-1 as templates, a 1.02-kb EcoRI-XbaI fragment (Product 3-1) comprising the following components: vegC promoter, lac operator, RBS of B. subtilis, coding sequences for intein Ssp DnaB and bFGF was obtained. Last of all, Product 3-1 was digested with EcoRI and XbaI, followed by ligation with a B. subtilis/E. coli shuttle vector, pM2-Veg (Lam et al. 1998) that had been digested with the same two restriction enzymes to result in construct pM2-DnaB-bFGF.

To construct expression construct pM2-CellBD-DnaB-bFGF, overlap extension PCR was performed, using oligos P1, P6, and P7-P10 (Table 1) as primers, and plasmids pFC and pM2-DnaB-bFGF (both described above) as templates. In doing so, the DNA sequence encoding the cellulose binding domain (CellBD) of an endoglucanase encoded by the cenA gene of C. fimi {GenBank:M15823.1; (Wong et al. 1986)} was cloned upstream of the DnaB-bFGF DNA fusion to obtain pM2-CellBD-DnaB-bFGF.

Protein Expression, Purification and Analysis of the bhFGF Product Shake Flask Cultivation

MMBL medium for growth of B. subtilis transformants in shake flasks was described previously (Sivakesava et al. 1999; Kwong et al. 2013b). To prepare seed cultures of the two transformants containing constructs pM2-DnaB-bFGF and pM2-CellBD-DnaB-bFGF, a fresh colony of each transformant was grown in 100 ml of MMBL medium supplemented with 20 μg ml⁻¹ of kanamycin. The culture was then grown at 250 rpm and 37° C. until the A₆₀₀ reading reached 8.0, followed by an addition of a final concentration of 0.5 mM IPTG to the growing cells. Culture samples were then collected at 2 h intervals for the analysis of bhFGF expression.

### Preparation of Cell Lysates

The cell pellets were each re-suspended in 120 μl of Tris-HCl buffer (50 mM, pH 8.0), followed by an addition of 83 μl of EDTA solution (0.25 M, pH 8.0) and incubation on ice for 5 min. The cells were then treated with 120 μl of lysozyme solution (10 mg ml⁻¹) at 37° C. for 20 min. To enhance cell lysis, 83 μl of solution X (10 mM EDTA, 10% Triton X-100, and 50 mM Tris-HCl, pH 8.0) was added, followed by gentle inversion of the tubes for 50 times. After centrifugation at 13,000 rpm for 10 min, lysate samples were collected and analyzed by Western blotting for bFGF expression as described previously. The images were quantified by densitometry using the ImageJ software (National Institutes of Health, USA).

### Fed-Batch Fermentation

To prepare the seed culture, B. subtilis [pM2-CellBD-DnaB-bFGF] cells were grown in MMBL medium supplemented with 20 μg ml⁻¹ of kanamycin at 250 rpm and 37° C. until the A₆₀₀ reading reached 1.0. Afterwards, 15 ml of the seed culture was transferred to a 500 ml Erlenmeyer flask containing 135 ml of fresh MMBL medium supplemented with 20 μg ml⁻¹ of kanamycin, and the culture was further grown at 250 rpm and 37° C. until the A₆₀₀ reading was 1.0. The entire 150 ml of seed was added into a 2-L fermentor containing 1.35 L of MMBL medium supplemented with 20 μg ml⁻¹ of kanamycin, with an addition of 1M NaOH to maintain the pH of the culture at ˜6.8. When the glucose was depleted and the pH began to increase, the culture was fed at 30 min intervals with 2 ml of 50% glucose. The speed of the impeller was set at 600 rpm to help improve pO₂ in the culture. When the pO₂ value dropped to a level of about 30%, a mixture of compressed air and pure oxygen adjusted at a ratio of 50/50 was used to improve pO₂ in the fermentor. The feeding was continued until the A₆₀₀ reading reached 20. When the pH of the culture turned to 6.8, it was induced with a final concentration of 0.5 mM IPTG. Culture samples were then collected at 2 h intervals for the analysis of bFGF expression.

Purification and Amino Acid Sequencing of bFGF

Heparin-agarose chromatography was employed to purify bFGF present in cell lysates as described previously. Purified bFGF was visualized on a SDS-PAGE gel stained with Coomassie blue. A band containing bFGF retrieved from the gel was analyzed by LC-mass spectrometry as described previously (Kwong et al. 2013a).

Bioactivity Determination of bFGF

The bioactivity of bFGF was measured based on its mitogenic effect on the proliferation of BALB/c 3T3 fibroblast cells employing the MTT assay as described previously. Interaction between bFGF and its receptors will activate intracellular signal transduction pathways, thereby phosphorylating fibroblast growth factor receptor substrate 2α (FRS2α). Phosphorylated FRS2α was detected using Western blotting; thus the assay enables the analysis and quantification of the mitogenic effect of recombinant bFGF.

### Results

### Rationale for the Development of the Expression Plasmids

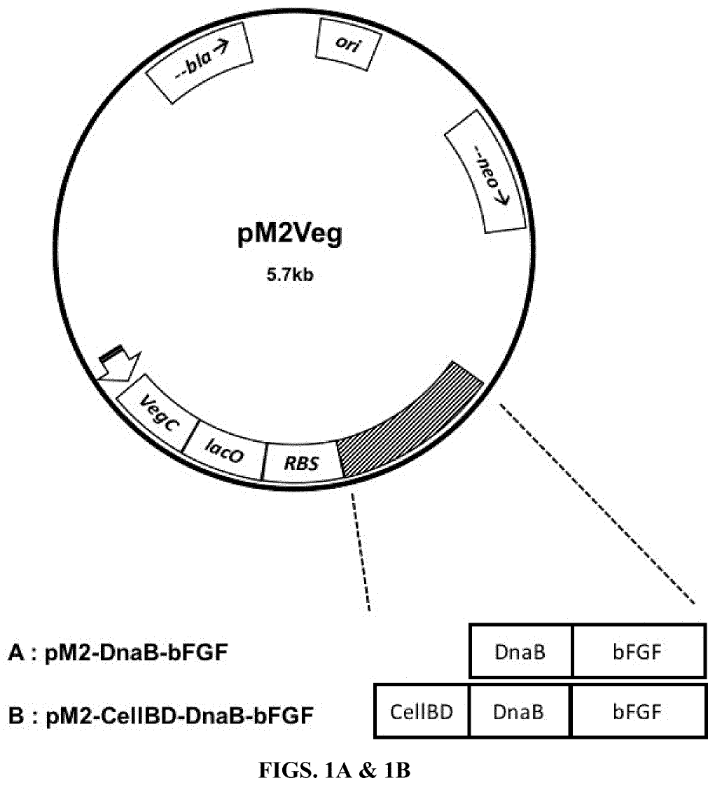

Since not much is known regarding how inteins operate in B. subtilis, it was reckoned that our experience gained in intein-mediated expression of heterologous proteins in E. coli might shed light on the engineering of self-cleavable fusions formed between inteins and target proteins in B. subtilis. From previous exercises, it was noted that the presence of both the N- and C-exteins embracing an intein could somehow enhance the success in achieving expression of a soluble fusion product formed between an intein and a foreign protein, irrespective of whether the target protein was expressed at the N- or C-terminus of the intein. To facilitate the recovery of the resulting intermediate fusion, which was hopefully retrievable using a facile protocol, it was decided that an 11 kD cellulose binding domain (CellBD) of an endoglucanase (Eng) encoded by the C. fimi cenA gene was exploited. Since CellBD was able to bind to cellulose and it was located at the N-terminal portion of Eng, it was expected that it might be expressed and act as an N-extein to provide the required anchorage for retrieving the intermediate product. Thus, in attempting to obtain bFGF expression mediated by the candidate intein, DnaB, two constructs, designated pM2-DnaB-bFGF (6.5 kb), which lacked the DNA coding sequence for CellBD (FIG. 1), and pM2-CellBD-DnaB-bFGF (6.8 kb), which harbored the DNA sequence concerned (FIG. 1), were engineered for the study.

Expression of bFGF in B. subtilis

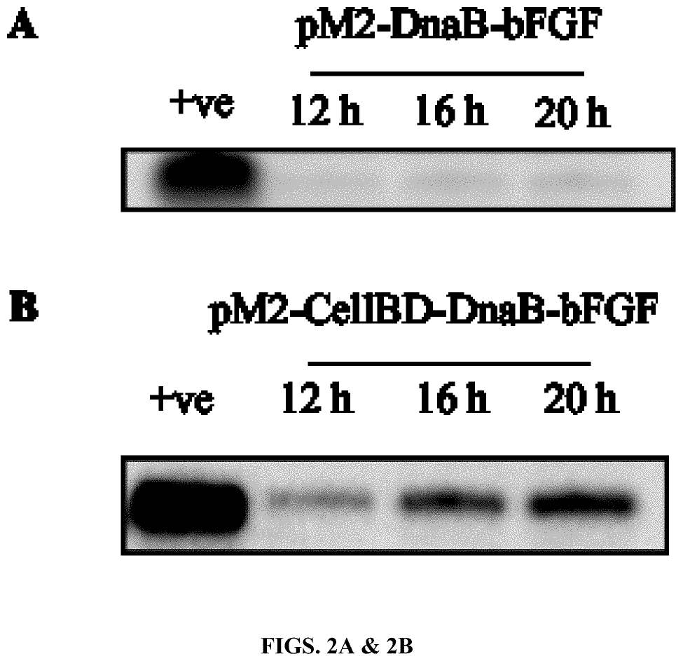

Results of time course experiments (FIG. 2) showed that construct pM2-DnaB-bFGF (FIG. 1) expressed only low levels of bFGF derived from cleavages of precursor/intermediate products (P/I). Despite the weak expression, the auto-cleavable activities appeared to be efficient, thus leaving almost undetectable trace of P/I (data not shown). It was then recollected that the presence of “an N-extein” such as EGF might enable P/I to achieve extendable and cleavable structures, as it was shown previously that EGF facilitated EGF-VMA-bFGF to achieve efficient expression and subsequent auto-processing to obtain authentic bFGF as the final product in E. coli. However, the same approach of employing EGF to result in the same P/I: EGF-VMA-bFGF, did not result in successful expression of bFGF in B. subtilis (data not shown). It was then considered that another pair of N-extein-intein fusion might yield better results. The 11 kDa CellBD, which was twice as big as EGF and was applied previously as a fusion tag (Greenwood et al. 1994), was considered for use to replace EGF. With the employment of CellBD, protein expression was expected to yield CellBD-DnaB-bFGF as P/I. Encouragingly, the replacement of CellBD-DnaB for EGF-VMA resulted positively in bFGF expression. In this connection, much higher levels of bFGF were detected in culture samples of B. subtilis [pM2-CellBD-DnaB-bFGF] transformant than those of its counterpart harboring construct pM2-DnaB-bFGF, which lacked the coding sequence for CellBD.

Time Course Expression of bFGF

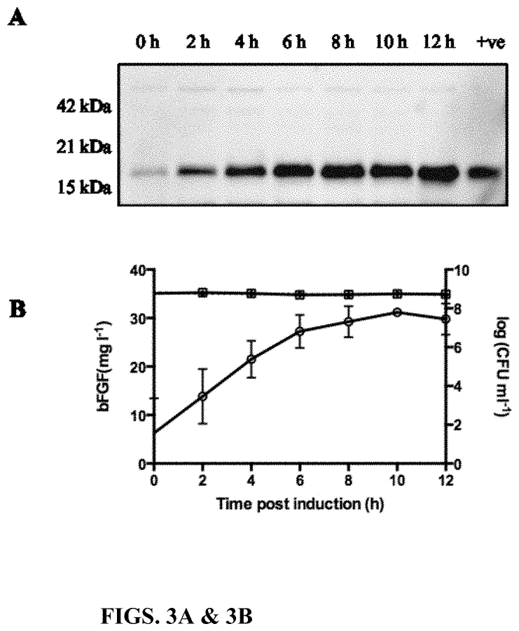

Results from initial shake flask cultivation supported that construct pM2-CellBD-DnaB-bFGF was able to keep the bFGF specific activity at high level when expression was achieved under induction (FIG. 2B). Time course experiments were then undertaken to obtain a more complete picture of bFGF production resulting from induced expression of pM2-CellBD-DnaB-bFGF in B. subtilis. Two pieces of useful information were obtained from the study. First, IPTG induction worked well to provide not only increasing but also a fruitful expression of up to 31 mg L⁻¹ of bFGF (FIG. 3). Second, this approach of expression resulted in also improved levels of bFGF specific activity (FIG. 3B). Apparently, both the stable cell growth of B. subtilis [pM2-CellBD-DnaB-bFGF] and the lack of plasmid curing (FIG. 3 B) contributed much to keep the bFGF specific activity at a high level.

Fermentative Production of bFGF

It was expected that improved levels of dissolved oxygen provided in the culture medium of B. subtilis, which is a strictly aerobic bacterium, would lead to enhanced cell growth of it, and hence higher yields of the target recombinant product. In view that MMBL medium and fed-batch fermentation conditions worked well for intein-mediated expression of recombinant proteins in E. coli (Kwong et al. 2016b), the same methodology was adopted to investigate the efficacy of scale-up expression of bFGF mediated by construct pM2-CellBD-DnaB-bFGF in B. subtilis cultivated in 2L fermentors.

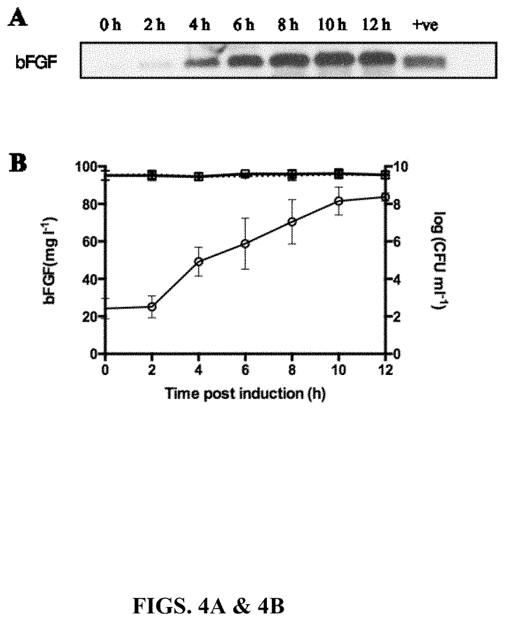

The results of the fermentation study showed remarkable improvements in the levels of both bFGF expression and cell density of the B. subtilis [pM2-CellBD-DnaB-bFGF] culture. The maximum yield of bFGF increased from 31 mg L⁻¹, attainable from shake flask cultivation (FIG. 3), to 84 mg L⁻¹, resulting from fermentative production (FIG. 4). Moreover, there was a 5-fold increase in the final cell density of the culture in scaling up from shake flask cultivation (FIG. 3) to small scale fermentation (FIG. 4). Presumably due to improvements in oxygen supply, mode of feeding of the cell culture, and refined pH of the growth medium, the B. subtilis [pM2-CellBD-DnaB-bFGF] culture was grown to achieve a high cell density, reaching a value of nearly 10¹⁰ cells ml⁻¹, even when bFGF expression was carried out under IPTG induction (FIG. 4). This high density, which was six times as high as that attained from induced expression in shake flasks, resulted in a pretty impressive yield, 84 mg L⁻¹, of bFGF despite the absence of accelerated cell growth throughout the entire time course study. Although the cells employed for induced expression had apparently entered the stationary phase, they remained intact even when bFGF was actively expressed. More attractively, the pM2-CellBD-DnaB-bFGF construct was stably maintained in its host cells (FIG. 4). This observation was strikingly different from that displayed by E. coli transformants taking on fermentative expression of recombinant proteins, during which dramatic plasmid loss was detected.

The Primary Structure of bFGF Expressed in B. subtilis

Western blot analysis (FIG. 4) revealed that recombinant bFGF retrieved from the lysate of Bacillus subtilis [pM2-CellBD-DnaB-bFGF] cells was shown to share the same molecular size with a 146 aa bFGF standard. However, it was not yet certain whether the bFGF product resulting from auto-cleavages of P/I possessed the exact 146 amino acid residues. A purified bFGF sample was then retrieved from the last time point of a fermentative growth and subjected to analysis by mass spectrometry (Kwong et at 2013a). The results of the analysis, for the first time, demonstrated that B. subtilis was able to undergo in vivo auto-cleavages of an intein fusion product: CellBD-DnaB-bFGF, to yield the desired product, bFGF, possessing the 146 aa authentic structure (Table 2).

Mitogenicity of Recombinant bFGF

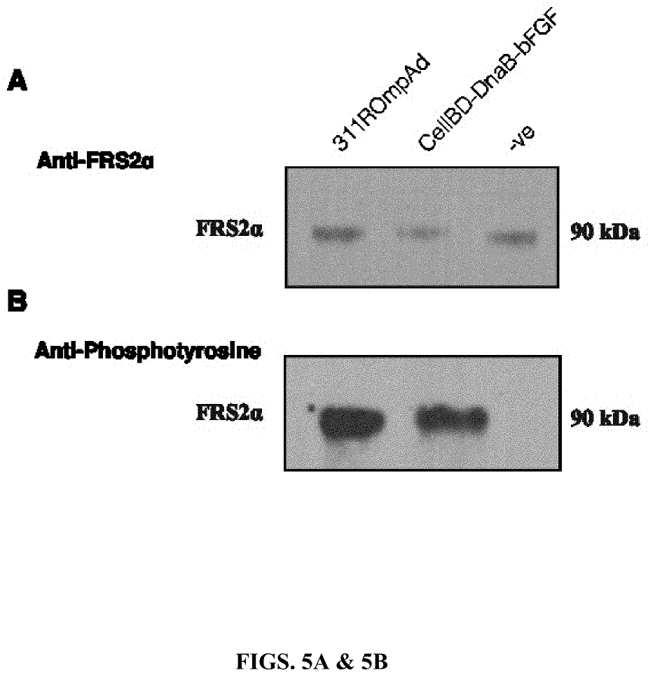

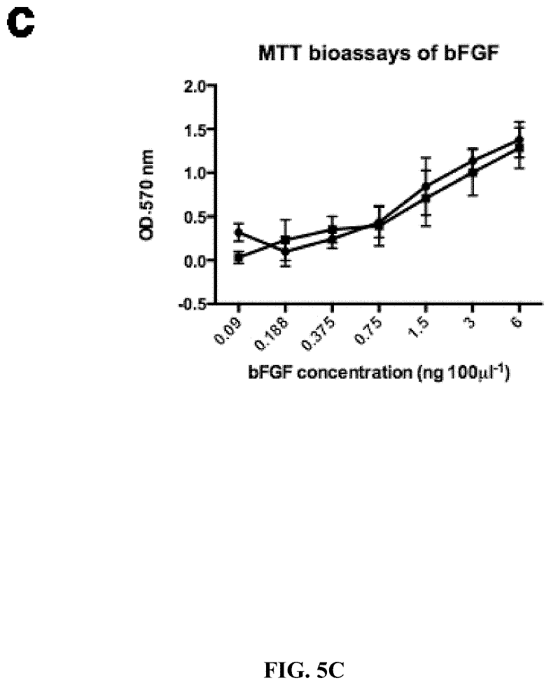

Reminiscent of the mitogenic effect demonstrated by authentic bFGF recovered from E. coli transformants (Kwong et al. 2013b; Kwong et al. 2016b), recombinant bFGF expressed by the intein DNA construct, pM2-CellBD-DnaB-bFGF, in B. subtilis was also shown to be biologically active (FIG. 5). Comparison between bFGF samples obtained from recombinant E. coli and B. subtilis cells revealed that they showed comparable potency (FIG. 5).

### Discussion

Since the first intein was discovered in the late 1980s, over 600 putative intein genes have been discovered. The cloning and characterization of intein genes has not only enabled us to better understand the molecular and biochemical functions of inteins, but also facilitate our employment of them to mediate expression of recombinant proteins with structures possessing the expected aa compositions. E. coli, being the most common host for heterologous gene expression due to its short doubling time, well known genetics, ease of handling and relatively inexpensive cost of production, has been the mostly preferred organism for recombinant protein expression mediated by inteins. Recently, E. coli systems employing inteins to mediate protein expression, which was accompanied by either in vivo or in vitro methods of auto-catalytic cleavage of fusions formed between target proteins and inteins, have been progressively developed. Notwithstanding E. coli is endowed with the aforesaid abilities, the fact that its employment as the host could result in the formation of inclusion bodies, plasmid curing, and dramatic cell death during recombinant protein expression, may impose difficulties on scale-up production.

The gram-positive bacterium, B. subtilis, being also well characterized, easily manipulated and relatively cheap to grow, is the second common host of choice employed for gene expression and has been engineered to express widely different secretory proteins. Nevertheless, despite being employed to achieve high levels of homologous protein expression, e.g., α-amylase, which has been expressed to result in over 1 g L⁻¹ of secreted product, attaining the same level of production of heterologous proteins in B. subtilis was shown difficult if not impossible.

Until the present invention, not much is known regarding how inteins operate in B. subtilis. The results from our study of fusions engineered among CellBD, DnaB and bFGF clearly demonstrated the success in having bFGF expressed as an intracellular, soluble and precisely cleaved product (FIG. 2). Our findings further support the idea that the intracellular compartment, cytoplasm, of B. subtilis, furnished an environment possessing the required conditions, similarly as its counterpart did in E. coli, to facilitate expression of heterologous proteins as soluble and auto-catalytically processed products. Although the use of DnaB alone resulted in only weak expression of bFGF, quite unexpectedly, by adding the 11 kD CellBD of the endoglucanase encoded by the cenA gene of C. fimi to the N-terminus of DnaB, expression of bFGF was highly enhanced (FIG. 3).

The application of CellBD as an anchor to the purification of fusion proteins expressed in E. coli was previously reported. Thus, it was envisaged that if our study resulted unfortunately in an insoluble CellBD-DnaB-bFGF P/I, CellBD would be useful for purifying the fusion protein, followed by its cleavage with the help of in vitro manipulations. Encouragingly, the described fusion approach resulted not only in successful expression of CellBD-DnaB-bFGF in the cytoplasm, but also a precursor protein that was soluble and auto-cleavable to yield bFGF possessing the 146 aa authentic structure (Table 2) as the product.

### REFERENCES

Akita, Sadanori, Kozo Akino, and Akiyoshi Hirano (2013) Basic fibroblast growth factor in scarless wound healing, Advances in wound care, 2: 44-49.
Akita, Sadanori, Kozo Akino, Toshifumi Imaizumi, and Akiyoshi Hirano (2008) Basic fibroblast growth factor accelerates and improves second-degree burn wound healing, Wound repair and regeneration, 16: 635-41.
Amitai, G., B. P. Callahan, M. J. Stanger, G. Belfort, and M. Belfort (2009) Modulation of intein activity by its neighboring extein substrates, Proc Natl Acad Sci USA, 106: 11005-10.
Baneyx, Francois, and Mirna Mujacic (2004) Recombinant protein folding and misfolding in Escherichia coli, Nat Biotech, 22: 1399-408.
Bentley, W. E., N. Mirjalili, D. C. Andersen, R. H. Davis, and D. S. Kompala (2009) Plasmid-encoded protein: the principal factor in the “metabolic burden” associated with recombinant bacteria. Biotechnology Bioengineering, 1990, Biotechnol Bioeng, 102: 1284-97; discussion 83.
Chen, Jingqi, Gang Fu, Yuanming Gai, Ping Zheng, Dawei Zhang, and Jianping Wen (2015) Combinatorial Sec pathway analysis for improved heterologous protein secretion in Bacillus subtilis: identification of bottlenecks by systematic gene overexpression, Microb Cell Fact, 14: 92.
Cottingham, I. R., A. Millar, E. Emslie, A. Colman, A. E. Schnieke, and C. McKee (2001) A method for the amidation of recombinant peptides expressed as intein fusion proteins in Escherichia coli, Nat Biotechnol, 19: 974-7.
Din, N., H. G. Damude, N. R. Gilkes, R. C. Miller, Jr., R. A. Warren, and D. G. Kilburn (1994) C1-Cx revisited: intramolecular synergism in a cellulase, Proc Natl Acad Sci USA, 91: 11383-7.
Elleuche, S., and S. Poggeler (2010) Inteins, valuable genetic elements in molecular biology and biotechnology, Appl Microbiol Biotechnol, 87: 479-89.
Esipov, R. S., D. A. Makarov, V. N. Stepanenko, M. A. Kostromina, T. I. Muravyova, Y. A. Andreev, I. A. Dyachenko, S. A. Kozlov, and E. V. Grishin (2017) Pilot production of the recombinant peptide toxin of Heteractis crispa as a potential analgesic by intein-mediated technology, Protein Expr Purif, 145: 71-76.
Fu, Z. B., K. L. Ng, C. C. Lam, K. C. Leung, W. H. Yip, and W. K. Wong (2006) A two-stage refinement approach for the enhancement of excretory production of an exoglucanase from Escherichia coli, Protein Expr Purif, 48: 205-14.
Fu, Z. B., K. L. Ng, T. L. Lam, and W. K. Wong (2005) Cell death caused by hyper-expression of a secretory exoglucanase in Escherichia coli, Protein Expr Purif, 42: 67-77.
Gemini (2018) Human Basic Fibroblast Growth Factor-147 (bFGF-147), https://www.gembio.com/product/human-basic-fibroblast-growth-factor-147-bfgf-147. Accessed 7 Feb. 2018.
Greenwood, J. M., N. R. Gilkes, R. C. Miller, Jr., D. G. Kilburn, and R. A. Warren (1994) Purification and processing of cellulose-binding domain-alkaline phosphatase fusion proteins, Biotechnol Bioeng, 44: 1295-305.
He, Q., A. Y. Fu, and T. J. Li (2015) Expression and one-step purification of the antimicrobial peptide cathelicidin-BF using the intein system in Bacillus subtilis, J Ind Microbiol Biotechnol, 42: 647-53.
He, W., W. Mu, B. Jiang, X. Yan, and T. Zhang (2016) Construction of a Food Grade Recombinant Bacillus subtilis Based on Replicative Plasmids with an Auxotrophic Marker for Biotransformation of d-Fructose to d-Allulose, J Agric Food Chem, 64: 3243-50.
Hirata, R., Y. Ohsumk, A. Nakano, H. Kawasaki, K. Suzuki, and Y. Anraku (1990) Molecular structure of a gene, VMA1, encoding the catalytic subunit of H(+)-translocating adenosine triphosphatase from vacuolar membranes of Saccharomyces cerevisiae, J Biol Chem, 265: 6726-33.
Hoppenreijs, V P, Elisabeth Pels, G F Vrensen, and W Frits Treffers (1994) Basic fibroblast growth factor stimulates corneal endothelial cell growth and endothelial wound healing of human corneas, Investigative ophthalmology & visual science, 35: 931-44.
Ikawa, Kaori, Hiroyuki Araki, Yukiharu Tsujino, Yasuhiro Hayashi, Kazuaki Igarashi, Yuji Hatada, Hiroshi Hagihara, Tadahiro OzAwA, Katsuya Ozaki, and Tohru Kobayashi (1998) Hyperexpression of the gene for a Bacillus α-amylase in Bacillus subtilis cells: enzymatic properties and crystallization of the recombinant enzyme, Bioscience, biotechnology, and biochemistry, 62: 1720-25.
Ingham, A. B., K. W. Sproat, M. L. Tizard, and R. J. Moore (2005) A versatile system for the expression of nonmodified bacteriocins in Escherichia coli, J Appl Microbiol, 98: 676-83.
Jacobs, M. F., J. B. Andersen, T. V. Borchert, and V. P. Kontinen (1995) Identification of a Bacillus subtilis secretion mutant using a beta-galactosidase screening procedure, Microbiology, 141 (Pt 7): 1771-9.
Kang, Z., S. Yang, G. Du, and J. Chen (2014) Molecular engineering of secretory machinery components for high-level secretion of proteins in Bacillus species, J Ind Microbiol Biotechnol, 41: 1599-607.
Krejci, E, Z Pesevski, O Nanka, and D Sedmera (2016) Physiological Role of FGF Signaling in Growth and Remodeling of Developing Cardiovascular System, Physiological Research, 65: 425.
Kurland, C. G., and H. Dong (1996) Bacterial growth inhibition by overproduction of protein, Mol Microbiol, 21: 1-4.
Kwon, Eun-Yeong, Kyung Mi Kim, Mi Kyoung Kim, In Young Lee, and Beom Soo Kim (2011) Production of nattokinase by high cell density fed-batch culture of Bacillus subtilis, Bioprocess and Biosystems Engineering, 34: 789-93.
Kwong, K. W., A. K. Ng, and W. K. Wong (2016a) Engineering versatile protein expression systems mediated by inteins in Escherichia coli, Appl Microbiol Biotechnol, 100: 255-62.
Kwong, K. W., K. L. Ng, C. C. Lam, Y. Y. Wang, and W. K. Wong (2013a) Authentic human basic fibroblast growth factor produced by secretion in Bacillus subtilis, Appl Microbiol Biotechnol, 97: 6803-11.
Kwong, K. W., T. Sivakumar, and W. K. Wong (2016b) Intein mediated hyper-production of authentic human basic fibroblast growth factor in Escherichia coli, Sci Rep, 6: 33948.
Kwong, K. W., and W. K. Wong (2013b) A revolutionary approach facilitating co-expression of authentic human epidermal growth factor and basic fibroblast growth factor in both cytoplasm and culture medium of Escherichia coli, Appl Microbiol Biotechnol, 97: 9071-80.
Lam, K. H., K. C. Chow, and W. K. Wong (1998) Construction of an efficient Bacillus subtilis system for extracellular production of heterologous proteins, J Biotechnol, 63: 167-77.
Lam, Tin-Long, Raymond S C Wong, and Wan-Keung R Wong (1997) Enhancement of extracellular production of a Cellulomonas fimi exoglucanase in Escherichia coli by the reduction of promoter strength, Enzyme and microbial technology, 20: 482-88.
Li, Weifen, Xuxia Zhou, and Ping Lu (2004) Bottlenecks in the expression and secretion of heterologous proteins in Bacillus subtilis, Research in microbiology, 155: 605-10.
Ng Alan, K L, C C Lam Gigi, W Y Kwong Keith, W N Dik Duncan, C Y Lai Nelson, L W Au Yong Jonathan, P Y Qian, and W K R Wong (2016) Enhancement of fish growth employing feed supplemented with recombinant fish growth hormone expressed in Bacillus subtilis, Research Journal of Biotechnology Vol, 11: 1.
Nijland, R., and O. P. Kuipers (2008) Optimization of protein secretion by Bacillus subtilis, Recent Pat Biotechnol, 2: 79-87.
Okabe, Keisuke, Ruka Hayashi, Noriko Aramaki-Hattori, Yoshiaki Sakamoto, and Kazuo Kishi (2013) Wound treatment using growth factors, Modern Plastic Surgery, 3: 108.
Palva, Ilkka (1982) Molecular cloning of α-amylase gene from Bacillus amyloliquefaciens and its expression in B. subtilis, Gene, 19: 81-87.
Peprotech (2018) Recombinant Human FGF-basic (154 a.a.) https://www.peprotech.com/peprogmp-recombinant-human-fgf-basic. Accessed 7 Feb. 2018.
Phan, T. T., H. D. Nguyen, and W. Schumann (2012) Development of a strong intracellular expression system for Bacillus subtilis by optimizing promoter elements, J Biotechnol, 157: 167-72.
Prospec (2018) FGF 2 HUMAN, https://www.prospecbio.com/fgf-2_human/. Accessed 7 Feb. 2018.
Saida, F., M. Uzan, B. Odaert, and F. Bontems (2006) Expression of highly toxic genes in E. coli: special strategies and genetic tools, Curr Protein Pept Sci, 7: 47-56.
Setrerrahmane, S., Y. Zhang, G. Dai, J. Lv, and S. Tan (2014) Efficient production of native lunasin with correct N-terminal processing by using the pH-induced self-cleavable Ssp DnaB mini-intein system in Escherichia coli, Appl Biochem Biotechnol, 174: 612-22.
Shah, N. H., and T. W. Muir (2014) Inteins: Nature's Gift to Protein Chemists, Chem Sci, 5: 446-61.
Shenandoah (2018) Human FGF-basic 154 aa (FGF2), https://www.shenandoah-bt.com/Human_FGF-basic_154.html. Accessed 7 Feb. 2018.
Sivakesava, S, Z N Xu, Y H Chen, J Hackett, R C Huang, E Lam, T L Lam, K L Siu, Raymond S C Wong, and Wan-Keung R Wong (1999) Production of excreted human epidermal growth factor (hEGF) by an efficient recombinant Escherichia coli system, Process biochemistry, 34: 893-900.
Starokadomskyy, P L, O V Okunev, D M Irodov, and V A Kordium (2008) Utilization of protein splicing for purification of the human growth hormone, Molecular biology, 42: 966-72.
Taguchi, Seiichi, Toshihiko Ooi, Kouhei Mizuno, and Hiromi Matsusaki (2015) Advances and needs for endotoxin-free production strains, Appl Microbiol Biotechnol, 99: 9349-60.
Terpe, Kay (2006) Overview of bacterial expression systems for heterologous protein production: from molecular and biochemical fundamentals to commercial systems, Appl Microbiol Biotechnol, 72: 211.
Unger, Ellis F, Lino Goncalves, Stephen E Epstein, Emily Y Chew, Carol Braun Trapnell, Richard O Cannon, Arshed A Quyyumi, Francis Loscalzo, and Jonathan A Stiber (2000) Effects of a single intracoronary injection of basic fibroblast growth factor in stable angina pectoris, The American journal of cardiology, 85: 1414-19.
Vicario-Abejón, Carlos, Karl K Johe, Thomas G Hazel, Diana Collazo, and Ronald D G McKay (1995) Functions of basic fibroblast growth factor and neurotrophins in the differentiation of hippocampal neurons, Neuron, 15: 105-14.
Wan, W., D. Wang, X. Gao, and J. Hong (2011) Expression of family 3 cellulose-binding module (CBM3) as an affinity tag for recombinant proteins in yeast, Appl Microbiol Biotechnol, 91: 789-98.
Wang, Xiaoyuan, and Peter J Quinn (2010) Endotoxins: lipopolysaccharides of gram-negative bacteria. Endotoxins: Structure, Function and Recognition (Springer).
Wang, Yule Yue, Z B Fu, K L Ng, Chui-Chi Lam, A K Chan, K F Sze, and W K R Wong (2011) Enhancement of excretory production of an exoglucanase from Escherichia coli with phage shock protein A (PspA) overexpression, J Microbiol Biotechnol, 21: 637-45.
Westers, L., H. Westers, and W. J. Quax (2004) Bacillus subtilis as cell factory for pharmaceutical proteins: a biotechnological approach to optimize the host organism, Biochim Biophys Acta, 1694: 299-310.
Wong, W. K., B. Gerhard, Z. M. Guo, D. G. Kilburn, A. J. Warren, and R. C. Miller, Jr. (1986) Characterization and structure of an endoglucanase gene cenA of Cellulomonas fimi, Gene, 44: 315-24.
Wu, W. Y., K. D. Miller, M. Coolbaugh, and D. W. Wood (2011) Intein-mediated one-step purification of Escherichia coli secreted human antibody fragments, Protein Expr Purif, 76: 221-8.
Yang, M., W. Zhang, S. Ji, P. Cao, Y. Chen, and X. Zhao (2013) Generation of an artificial double promoter for protein expression in Bacillus subtilis through a promoter trap system, PLoS One, 8: e56321.
Ye, Ruiqiong, June-Hyung Kim, Byung-Gee Kim, Steven Szarka, Elaine Sihota, and Sui-Lam Wong (1999) High-level secretory production of intact, biologically active staphylokinase from Bacillus subtilis, Biotechnol Bioeng, 62: 87-96.
Zhang, Yuejuan, Kun Zhang, Yi Wan, Jing Zi, Yan Wang, Jun Wang, Lili Wang, and Xiaochang Xue (2015) A pH-induced, intein-mediated expression and purification of recombinant human epidermal growth factor in Escherichia coli, Biotechnology progress, 31: 758-64.
Zhinan, Xu, Liu Gang, Cen Peilin, and W K R Wong (2000) Factors influencing excretive production of human epidermal growth factor (hEGF) with recombinant Escherichia coli K12 system, Bioprocess and Biosystems Engineering, 23: 669-74.

TABLE 1
Primers employed in this study

| Primer | Orientation | Sequence (5′ to 3′)* |
|---|---|---|
| P1: EcoR1 VegC F | Forward | AAAGAATTCTAATTTAAATTTTATTTG |
| (SEQ ID NO. 2) | | |
| P2: VegC DnaB R | Reverse | CATTTTTTATCACCTC |
| (SEQ ID NO. 3) | | |
| P3: VegC DnaB F | Forward | GAGGTGATAAAAAATGTGTATCTCTGGCGATAG |
| (SEQ ID NO. 4) | | |
| P4: DnaB-bFGF R | Reverse | CTCTGGCAAGGCTGGGTTGTGTACAATGAT |
| (SEQ ID NO. 5) | | |
| P5: DnaB-bFGF F | Forward | ATCATTGTACACAACCCAGCCTTGCCAGAG |
| (SEQ ID NO. 6) | | |
| P6: XbaI-bFGF R | Reverse | AATTTCTAGATCATTATTAGCTCTTAGCAGACATT |
| (SEQ ID NO. 7) | | |
| P7: VegC CellBD R | Reverse | GCAGCCGGGAGCCATTTTTATCACCTCCTT |
| (SEQ ID NO. 8) | | |
| P8: VegC CellBD F | Forward | AAGGAGGTGATAAAAATGGCTCCCGGCTGC |
| (SEQ ID NO. 9) | | |
| P9: CellBD-DnaB R | Reverse | ATCGCCAGAGATACAGGTGCCCGTGCAGGT |
| (SEQ ID NO. 10) | | |
| P10: CellBD-DnaB F | Forward | ACCTGCACGGGCACCTGTATCTCTGGCGAT |
| (SEQ ID NO. 11) | | |

*Restriction sites used in the cloning experiments are underlined.

TABLE 2
Analysis of purified bFGF by liquid chromatography tandem mass
spectrometry

| | | | | Ion |
|---|---|---|---|---|
| Constructs | Peptide^{a} | Mr(Calc)^{b} | Mr(Expt)^{c} | Score |
| CellBD-DnaB-bFGF | -PALPEDGGSGAFPPGHFKD | 1779.858 | 1780.0282 | 40 |
| | (SEQ ID NO. 12) | | | |
| | KAILFLPMSAKS- | 1105.6205 | 1105.9454 | 59 |
| | (SEQ ID NO. 13) | | | |

^{a}subsequent to partial trypsin digestion of bFGF, the N-terminal and C-terminal sequences were identified by the Mascot search engine.
^{b}Theoretical mass-to-charge ratio of the peptide
^{c}Experimental mass-to-charge ratio of the peptide

1. A method of production of authentic and bioactive human basic fibroblast growth factor (bFGF) of 146 amino acids intracellularly and without any modification at either C- or N-terminal of the bFGF, comprising the steps of providing a Bacillus subtilis host and introducing a DNA construct into the Bacillus subtilis host to produce a transformed Bacillus subtilis host, wherein the DNA construct including an insert having, from 5′ to 3′, a cellulose binding domain (CellBD), an intein sequence and a DNA coding for the bFGF polypeptide, and subjecting the transformed Bacillus subtilis host to a shake flask cultivation process or a fed-batch fermentation process, whereby to enable the transformed Bacillus subtilis host to produce the bFGF in a soluble form, cleaved, independent from proteins encoded by DNA regions preceding and subsequent to the bFGF DNA coding in the insert, and intracellularly.

2. A method as claimed in claim 1, wherein the intein sequence is Ssp DnaB.

3. A method as claimed in claim 1, wherein when subjected to the shake flask cultivation process, comprising a step of using a shake flask with a 100 ml of culturing medium; or when subjected to the fed-batch fermentation process, comprising a step of using a fermenter with a 2-L of culturing medium.

4. A method as claimed in claim 3, wherein the production of the bFGF via the fed-batch fermentation process is more than twice as much as via the shake flask cultivation process.

5. A biological system engineered from a Bacillus subtilis host, comprising a DNA construct including an insert comprising, from 5′ to 3′, a cellulose binding domain (CellBD), an intein sequence and a DNA coding for the bFGF polypeptide.

6. A system as claimed in claim 5, wherein the intein sequence is Ssp DnaB.

7. A DNA construct comprising an insert comprising, from 5′ to 3′, a cellulose binding domain (CellBD), an intein sequence and a DNA coding for a bFGF polypeptide, wherein the bFGF polypeptide is an authentic and bioactive human basic fibroblast growth factor of 146 amino acids intracellularly and without any modification at either C- or N-terminal of the bFGF.

8. A DNA construct as claimed in claim 7, wherein the intein sequence is Ssp DnaB.


SEQUENCE LISTING (13 sequences)

SEQ ID NO: 1 - protein, 146 aa, Homo sapiens
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 2 - DNA, 27 bp, Artificial Sequence
  Description of Artificial Sequence Synthetic primer
aaagaattctaatttaaattttatttg

SEQ ID NO: 3 - DNA, 16 bp, Artificial Sequence
  Description of Artificial Sequence Synthetic primer
cattttttatcacctc

SEQ ID NO: 4 - DNA, 33 bp, Artificial Sequence
  Description of Artificial Sequence Synthetic primer
gaggtgataaaaaatgtgtatctctggcgatag

SEQ ID NO: 5 - DNA, 30 bp, Artificial Sequence
  Description of Artificial Sequence Synthetic primer
ctctggcaaggctgggttgtgtacaatgat

SEQ ID NO: 6 - DNA, 30 bp, Artificial Sequence
  Description of Artificial Sequence Synthetic primer
atcattgtacacaacccagccttgccagag

SEQ ID NO: 7 - DNA, 35 bp, Artificial Sequence
  Description of Artificial Sequence Synthetic primer
aatttctagatcattattagctcttagcagacatt

SEQ ID NO: 8 - DNA, 30 bp, Artificial Sequence
  Description of Artificial Sequence Synthetic primer
gcagccgggagccatttttatcacctcctt

SEQ ID NO: 9 - DNA, 30 bp, Artificial Sequence
  Description of Artificial Sequence Synthetic primer
aaggaggtgataaaaatggctcccggctgc

SEQ ID NO: 10 - DNA, 30 bp, Artificial Sequence
  Description of Artificial Sequence Synthetic primer
atcgccagagatacaggtgcccgtgcaggt

SEQ ID NO: 11 - DNA, 30 bp, Artificial Sequence
  Description of Artificial Sequence Synthetic primer
acctgcacgggcacctgtatctctggcgat

SEQ ID NO: 12 - protein, 19 aa, Homo sapiens
PALPEDGGSGAFPPGHFKD

SEQ ID NO: 13 - protein, 12 aa, Homo sapiens
KAILFLPMSAKS